SEQUENCE LISTING (17 sequences)

SEQ ID NO: 1 - protein, 15 aa, Synthetic construct
GLNDIFEAQKIEWHE

SEQ ID NO: 2 - protein, 26 aa, synthetic construct
KRRWKKNFIAVSAANRFKKISSSGAL

SEQ ID NO: 3 - protein, 8 aa, synthetic construct
DYKDDDDK

SEQ ID NO: 4 - protein, 9 aa, synthetic construct
YPYDVPDYA

SEQ ID NO: 5 - protein, 6 aa, synthetic construct
HHHHHH

SEQ ID NO: 6 - protein, 10 aa, synthetic construct
EQKLISEEDL

SEQ ID NO: 7 - protein, 15 aa, synthetic construct
KETAAAKFERQHMDS

SEQ ID NO: 8 - protein, 38 aa, synthetic construct
MDEKTTGWRGGHVVEGLAGELEQLRARLEHHPQGQREP

SEQ ID NO: 9 - protein, 13 aa, synthetic construct
SLAELLNAGLGGS

SEQ ID NO: 10 - protein, 8 aa, synthetic construct
TQDPSRVG

SEQ ID NO: 11 - protein, 14 aa, synthetic construct
GKPIPNPLLGLDST

SEQ ID NO: 12 - protein, 8 aa, synthetic construct
DLYDDDDK

SEQ ID NO: 13 - protein, 8 aa, synthetic construct
WSHPQFEK

SEQ ID NO: 14 - protein, 590 aa, Homo sapiens
SPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIESNKAIQDPRLFAEE

SEQ ID NO: 15 - protein, 612 aa, synthetic construct
HHHHHHSSGVDLGTENLYFQSNASPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEE

SEQ ID NO: 16 - protein, 613 aa, synthetic construct
AHHHHHHSSGVDLGTENLYFQSNASPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEE

SEQ ID NO: 17 - protein, 634 aa, synthetic construct
MKFLVNVALVFMVVYISYIYAAHHHHHHSSGVDLGTENLYFQSNASPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEE

(19) United States
(12) Patent Application Publication
(10) Pub. No.: US 2025/0326821 A1
(43) Pub. Date: Oct. 23, 2025

(54) PROCESS FOR PREPARATION OF SECRETORY IgA AND SECRETORY IgM AND USE THEREOF FOR TREATING NECROTIZING ENTEROCOLITIS

(71) Applicants: Michael R. Simon, Ann Arbor, MI (US); William Clay Brown, Ann Arbor, MI (US)

(72) Inventors: Michael R. Simon, Ann Arbor, MI (US); William Clay Brown, Ann Arbor, MI (US)

(21) Appl. No.: 18/689,556

(22) PCT Filed: Jan. 8, 2024
(86) PCT No.: PCT/US2024/010639

§ 371 Date: Mar. 6, 2024

Related U.S. Application Data
(60) Provisional application No. 63/479,811, filed on Jan. 13, 2023.

(51) Int. Cl. C07K 16/06; A61K 39/00; A61P 1/00

(52) U.S. Cl. CPC C07K 16/06; A61P 1/00; A61K 2039/505; C07K 2317/94; C07K 2319/20

(57) ABSTRACT

A process for synthesizing and separating secretory IgA from a mixture of IgA monomer and IgA dimer is provided The process includes covalently binding affinity tagged or epitope tagged recombinant secretory component to the IgA dimer in the mixture and then binding the affinity tagged or an epitope tagged secretory IgA to immobilized moieties on the solid phase support resin to which the affinity tag or epitope tag binds and then eluting the affinity tagged or an epitope tagged secretory IgA with release buffer. A process for synthesizing and separating secretory IgM from a mixture of IgM and other plasma proteins is provided. A process is provided for inhibiting or preventing symptoms of necrotizing enterocolitis in a subject that includes the oral administration to the subject of a human polyclonal secretory IgA formed by the conjugation of human recombinant secretory component and pooled human plasma derived dimeric and polymeric.

### RELATED APPLICATIONS

This application claims priority benefit of U.S. Provisional Application Ser. No. 63/479,811 filed 13 Jan. 2023; the contents of which are hereby incorporated by reference.

### GOVERNMENT SUPPORT

This invention was made with government support under 1R44DK130749-01A1 awarded by the National Institutes of Health. The government has certain rights in the invention.

### FIELD OF THE INVENTION

This invention relates in general to a process for the preparation of human semisynthetic secretory IgA and secretory IgM containing recombinant human secretory component or molecular variations of human secretory component from recombinant human secretory component and IgA and/or IgM, and in particular to a process that is scalable to allow the production of commercial quantities of medicaments containing the same, the purpose of which includes the treatment of necrotizing enterocolitis.

### BACKGROUND OF THE INVENTION

Necrotizing enterocolitis is caused by inflammation of the intestines in premature infants. Necrotizing enterocolitis may be superficial, effecting only the mucosal lining of the gut, or may be so severe that the entire thickness of the intestinal wall is involved and there is perforation caused by the inflammation (Zani and Pierro, 2019).

The incidence of necrotizing enterocolitis is about 5% to 7% of premature infants born after fewer than 33 weeks gestation (Zani and Pierro, 2019) or weighing less than 1500 grams (Hackam and Caplan, 2018). Symptoms range from bradycardia to shock (Zani and Pierro 2019). Maternal IgA in breast milk has been found to protect premature infants from necrotizing enterocolitis (Gopalakrishna, 2019; Hand. NIH published report NIH/R01-DK120697-01A1, 2020).

Probiotics have been found to contribute to prevention of this syndrome (Zani and Pierro, 2019). Medical management includes bowel rest by withholding feedings for bowel rest, and general support such as maintaining adequate ventilation, and tissue perfusion and blood pressure., and antibiotics as needed. (Zani and Pierro, 2019). In addition, maternal breast milk which contains secretory IgA has been found to be effective in prevention of necrotizing enterocolitis (Gopalakrishna et al., 2019). Breast milk contains secretory IgA which regulates the intestinal microbiome and facilitates intestinal homeostasis (Rogier, et al. 2014). Prospects for new treatments include hypothermia and stem cell therapy (Zani and Pierro, 2019). Oral human immunoglobulin treatment has also shown efficacy in treating necrotizing enterocolitis (Eibl et al 1988, Eibl et al 1990, Wolf and Eibl 1991). However, these authors did not use, or contemplate the oral use of, secretory immunoglobulins.

However, the prior art has failed to evaluate orally administered human polyclonal semisynthetic secretory IgA's comprised of recombinant human secretory component together with plasma derived polyclonal human IgA as a potential medicament for the treatment of necrotizing enterocolitis even if prospective usage thereof has been contemplated: EP 2636682. Prior art did not contemplate associated industrial manufacturing processes to assure high purity and high throughput that are presented herein.

Thus, there exists a need for IgA and IgM therapeutics that are resistant to gastrointestinal tract degradation. There also exists a need for a human polyclonal secretory IgA therapeutic for the treatment of necrotizing enterocolitis. There further exists a need to provide such a therapeutic in a dosing form well suited for treating an infected subject including infants. Human plasma derived IgA has been successfully combined with recombinant secretory component to produce secretory IgA with biological activity (Longet et al 2013) (Simon et al 2014) (Chiari et al. 2021).

### SUMMARY OF THE INVENTION

A process is provided for inhibiting symptoms of necrotizing enterocolitis in a subject suffering therefrom that includes the oral administration of semisynthetic polyclonal human secretory IgA to the subject with necrotizing enterocolitis or at increased risk of necrotizing enterocolitis. When administered in a therapeutic quantity based on the subject characteristics and the type of IgA, symptoms of necrotizing enterocolitis in that subject are inhibited. The administered immunoglobulin is readily formed from polyclonal sources and recombinant human secretory component.

This invention specifies an industrial method for the manufacture of polyclonal human secretory IgA (sIgA) which is not otherwise obtainable in amounts suitable for medicinal use. The sIgA is readily administered in a dimeric, or polymeric form that includes recombinant human secretory component. The recombinant human secretory component many include N-terminus modifications that promote efficient purification required for a medicament. The secretory component being modified to contain an affinity tag or an epitope tag to form secretory IgA and/or secretory IgM containing said affinity tag or epitope tag that is useful for capture by a solid phase support resin. The protein solution which now contains the affinity tagged or epitope-tagged secretory IgA or affinity tagged or epitope-tagged secretory IgM is then amenable to being applied to a solid phase support resin. The adherence of the exposed affinity-tagged or epitope-tagged secretory IgA or secretory IgM to the resin and the unwanted components flow through and are thus removed. The desired product, secretory IgA or secretory IgM is then eluted from the solid phase support resin using a release agent.

### BRIEF DESCRIPTION OF THE DRAWINGS

FIG. 1 is an ELISA showing (A) colostral secretory IgA (sIgA) and (I) synthesized sIgA, the ELISA plate coated with mouse anti-human secretory component antibody;

FIG. 2 is a space filling three-dimensional structure of a human IgA secretory component showing the native N-terminus extending therefrom in an easily accessible position (PDB 3cm9); and

FIG. 3 is an amino acid sequence of human IgA secretory component with N-terminus inclusion of a histidine tag and spacer, with an optional cap amino acid and cleaved signal sequence.

### DETAILED DESCRIPTION OF THE PREFERRED EMBODIMENTS

The present invention has utility for the preparation of secretory IgA or secretory IgM. In some inventive embodiments, the IgA is derived from a mixture of monomeric and dimeric plasma IgA, in another inventive embodiment it also contains other plasma proteins, and in yet other inventive embodiments, the preparation of secretory IgM is derived from a mixture of IgM with other plasma proteins. The present invention is superior to monomeric IgA or pentameric IgM administered orally because the presence of secretory component provides resistance to degradation and protects the IgA or IgM from digestion in the gastrointestinal tract (U.S. Pat. No. 9,932,392). Without intending to be bound to particular theory. it is believed that the increased efficacy of the present invention is achieved for secretory IgA or secretory IgM owing to the propensity of monomeric IgA and pentameric IgM to degrade in the gastrointestinal tract. The resultant dosing requirements decrease treatment costs using secretory IgA or IgM. While the present invention is further detailed principally with respect to IgA, it is appreciated that the process and medicaments that result are equally applicable to IgM and the resulting secretory IgM, regardless of whether the tag is retained or removed. Resort to N-terminus affinity-tagged secretory component further promotes purification and treatment efficiency.

The present invention also has utility as a treatment or prevention of necrotizing enterocolitis. The process of treatment or prevention includes treatment with polyclonal-secretory IgA that is, dimeric or polymeric and polyclonal. Polyclonal dimeric or polymeric IgA is recoverable from the plasma fractionation waste product Cohn fraction III precipitate or equivalent (Simon, 2014). It is also recoverable from the ion exchange plasma fractionation process used to recover other plasma proteins (U.S. Pat. No. 9,828,418B2, 10385117B2, and 9828418B2) or from an IgG recovery anion exchange column strip solution.

It is to be understood that in instances where a range of values are provided that the range is intended to encompass not only the end point values of the range but also intermediate values of the range as explicitly being included within the range and varying by the last significant figure in the range. By way of example, a recited range from 1 to 4 is intended to include 1-2, 1-3, 2-4, 3-4, and 1-4.

As used herein, a “subject” is defined as a human.

As used herein, “dimeric and polymeric IgA” is defined as a construct that contains two or more IgA monomers bonded to at least one joining (J) chain.

As the present invention uses an immunoglobulin rather than a metabolic or immunological inhibitor, an effective treatment or preventative is provided which does not otherwise disturb the body's normal metabolism.

The following description of the preferred embodiment(s) is merely exemplary in nature and is in no way intended to limit the invention, its application, or uses.

Secretory IgA molecules are polyclonal and dimeric or polymeric; and are all known to the art, as evidenced for example, by the references incorporated herein.

Allogeneic immunoglobulins administered directly to the gastrointestinal tract have minimal or no side effects because they are naturally present in the gastrointestinal tract. Dimeric and polymeric IgA according to the present invention is bound to recombinant human secretory component in order to mimic naturally secreted intestinal secretory IgA which is endogenous to the subject. The administration of the semisynthetic secretory IgA compensates for the absence of naturally secreted secretory IgA in breast milk which normally provides the secretory IgA in neonatal infants.

An affinity tag or an epitope tag that is efficacious for the present invention is one of: peptide tags: AviTag, a peptide allowing biotinylation by the enzyme BirA so the protein can be isolated by streptavidin; GLNDIFEAQKIEWHE (SEQ ID No. 1); calmodulin-tag, a peptide bound by the protein calmodulin KRRWKKNFIAVSAANRFKKISSSGAL (SEQ ID No. 2); FLAG-tag, a peptide recognized by an antibody DYKDDDDK (SEQ ID No. 3); Hemaglutinin-tag, a peptide recognized by an antibody YPYDVPDYA (SEQ ID No. 4); His-tag, 5-10 histidines bound by a nickel or cobalt or other divalent cation chelate HHHHHH (SEQ ID No. 5); Myc-tag, a short peptide recognized by an antibody EQKLISEEDL (SEQ ID No. 6); S-tag KETAAAKFERQHMDS (SEQ ID No. 7); SBP-tag, a peptide which binds to streptavidin MDEKTTGWRGGHVVEGLAGELEQLRARLEHHPQGQREP (SEQ ID No. 8); Softag 1, for mammalian expression SLAELLNAGLGGS (SEQ ID No. 9); Softag 3, for prokaryotic expression TQDPSRVG (SEQ ID No. 10); V5 tag, a peptide recognized by an antibody GKPIPNPLLGLDST (SEQ ID No. 11); Xpress tag DLYDDDDK (SEQ ID No. 12); Biotin Carboxyl Carrier Protein, a protein domain recognized by streptavidin; Glutathione-S-transferase-tag, a protein which binds to immobilized glutathione; Green fluorescent protein-tag, a protein which is spontaneously fluorescent and can be bound by nanobodies; Maltose binding protein-tag, a protein which binds to amylose agarose; Nus-tag; Strep-tag, a peptide which binds to streptavidin, or the modified streptavidin called streptactin Strep-tag II: WSHPQFEK (SEQ ID No. 13); Thioredoxin-tag; TC tag; or Ty tag.

Plasma IgA contains a mixture of monomer and dimer (Delacroix et al. 1981; Delacroix et al. 1983; Longet et al. 2013, Simon et al 2014). In some embodiments of the present invention, plasma dimeric IgA in the naturally occurring monomer-dimer mixture is covalently bound to the affinity peptide tagged secretory component in vitro. In other inventive embodiments, native secretory component is covalently bonded to one or more amino acid residues through conventional synthetic techniques (Hermanson GT 1996). As an example, using a histidine tag, it is appreciated that a single histidine residue or a poly histidine having typically between 2 and 20 histidine residues is added to N terminus of the secretory component, regardless of whether produced by recombinant, synthetic addition, or other technique. The secretory IgA is now histidine tagged by virtue of the divalent bonding of the histidine tagged recombinant secretory component to the naturally occurring IgA dimer, The novel method of obtaining purified secretory IgA that is thus formed is to remove the secretory IgA that is now tagged by affinity binding of one of the aforementioned tags to a binding moiety immobilized on a resin or by further example by a secretory component N terminus histidine tag immobilized on a nickel⁺² resin. Alternatively, other immobilized divalent metal ions such as cobalt, zinc, copper or iron can be used. Alternatively, a FLAG peptide is used in certain inventive embodiments and antibody to the FLAG peptide is immobilized on the solid support resin. FLAG tags have been detailed elsewhere, as for example, U.S. Pat. No. 4,703,004. The resultant secretory IgA has utility, for example, as a treatment of C. difficile associated diseases such as Clostridium difficile colitis, pseudomembranous colitis, antibiotic associated diarrhea, and in particular, to secretory immunoglobulin A (IgA) compositions administered in the form of pharmaceutical compositions. The above process is equally applicable to IgM to form purified secretory IgM.

In one embodiment, the invention provides a process for medical treatment of humans involving the oral administration of secretory IgA which can be derived from a number of sources. One such source for the IgA is pooled human plasma following Cohn cold ethanol fractionation to produce fraction III precipitate as performed by those of skill in the art of protein separation. The IgA byproduct is further purified by adsorption onto jackbean lectin (jacalin) and/or onto an ion exchange medium in neutral or slightly acidic conditions as performed by those of skill in the art of protein purification (Kabir S, 1998; and U.S. Pat. No. 9,828,418).

A more detailed description of an exemplary isolation of an IgA component as a byproduct from pooled human plasma or hyperimmune pooled human plasma is as follows. Ethanol fractionation of pooled human plasma is a well-known process to prepare immunoglobulin G. Pooled human plasma is first obtained from licensed plasmapheresis centers in the United States and tested for various pathogens including the HIV virus. The first manufacturing step of most commercial immunoglobulin G preparations involves a modified cold ethanol fractionation according to Cohn to produce Cohn fraction II. In the fractionation process, many infectious viruses are eliminated from the pooled human plasma. Following fractionation, the Cohn fraction II is subjected to adsorption onto an ion exchange medium. This step may selectively reduce the IgA concentration to less than 0.1%. Such a step is important for producing immunoglobulin G for intravenous infusion into humans. This is because some individuals undergo an anaphylactic-like reaction if treated with intravenous IgG that contains IgA as an impurity.

The modified cold ethanol fractionation process according to Cohn is a series of fractionations using various levels of ethanol, pH, and temperature to produce a fraction II which is further treated to produce immunoglobulins as described above. In the fractionation process, pooled human plasma is first treated to produce a cryoprecipitate and cryo-supernatant. Alternatively, it is appreciated that the source plasma may be autologous plasma or hyperimmune human plasma, either pooled, or from a single individual who has been immunized against a specific disease.

In another embodiment, the IgA component is be prepared from plasma by ion exchange chromatography.

In still another embodiment, the IgA component is prepared by hybridoma techniques to provide antigen-specific dimeric IgA. Hybridoma techniques are described originally in Kohler and Milstein, Nature 1975; 256:495-497 with more recent advances summarized in Berzofsky et al., Fundamental Immunology, Third Edition, 1993, pp 455-62.

Regardless of the source, the cryo-supernatant is subjected to a first ethanol fractionation to yield a supernatant I. Supernatant I is subjected to a second ethanol fractionation to yield fraction II+III. Fraction II+III is subjected to a third ethanol fractionation procedure to yield a supernatant III and Fraction III precipitate.

The fraction III precipitate enriched in IgA is generally discarded as an unwanted byproduct. According to the present invention, from this unwanted fraction III precipitate, IgA is obtained following ion exchange adsorption purification or affinity chromatographic purification is further treated by incubation with immobilized hydrolases to inactivate viruses and vasoactive substances. Such treatment has been proven to eliminate many viruses tested including HIV, Sindbis, and vaccinia. Other antiviral treatments, as known to those skilled in the art, are used in combination and consist of solvent detergent processes, nanofiltration and/or heat inactivation. Usually, three antiviral steps are implemented. Following incubation to remove viruses, the concentration of the active material is adjusted with sterile saline or buffered solutions to ensure a constant amount of active material per milliliter of reconstituted product. Finally, the solution with a constant amount of reconstituted product is sterilized by filtration before use.

The ethanol fractionation process according to Cohn is well known in the art and is described in Cohn et al., J. Am. Chem. Soc. 1946; 68:459-475, and in more detail in pages 576-602, Oncley et al., J. Am. Chem. Soc. 1949; 71:541-550, and in most detail in pages 576-602, Kirk-Othmer Encyclopedia of Chemical Technology, Vol. 3, second edition (1963). Alternatively, ion exchange chromatography may be used to obtain the dimeric and polymeric IgA byproduct during the manufacture of intravenous immunoglobulin. From 4% to 22% of plasma IgA is dimeric and polymeric IgA (Delacroix et al. 1981; Delacroix et al. 1983). The resulting dimeric IgA-J chains are purified to form a medicament. In specific embodiments, the compositions of the invention contain, in addition to the IgA component, recombinant human secretory component. Human secretory component can be produced by recombinant techniques as described in Crottet et al., 1999.

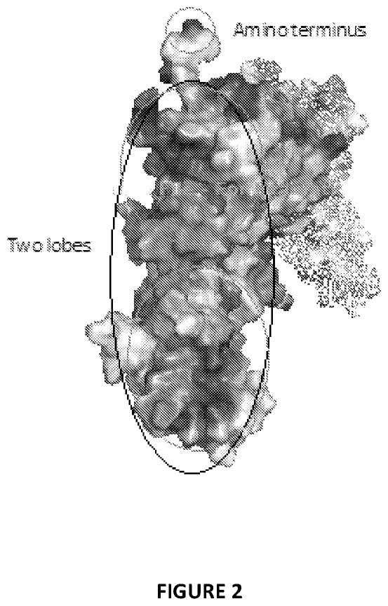

The dimeric and polymeric IgA present in the plasma IgA monomer-polymer mixture is further coupled to secretory component, which may be a human secretory component, that is recombinantly produced to include a N terminus histidine tag or another of the aforementioned tags; or subsequently covalently bonded to a peptide tag such as histidine or poly-histine oligopeptide. In specific inventive embodiments, dimeric and polymeric IgA according to the present invention is bound to recombinant human secretory component in order to mimic naturally secreted intestinal secretory IgA which is endogenous to the subject. The administration of the semisynthetic secretory IgA compensates for the absence of naturally secreted secretory IgA in breast milk which normally provides the secretory IgA. Human secretory component is shown graphically as a space-fill model in FIG. 2, with two lobes being noted therein and the steric exposure of the N-terminus (aminoterminus) extending therefrom being notable. According to some inventive embodiments, a polyhistidine tag is added and extends from the N-terminus, which points away from the bulk of the newly formed secretory immunoglobulin into the ambient solution and is accessible for metal-affinity resin chromatography. This steric exposure is readily exploited for efficient purification relative to a like C-terminus tag on the secretory component which is situated between the secretory component and the IgA dimer to which it is bound and is not exposed to the ambient solution.

In certain inventive embodiments, the coupling of IgA to secretory component is accomplished by forming disulfide bonds under mildly oxidizing conditions. (Jones R. M. L., Schweikart F., Frutiger S., Jaton J-C., Hughes G. J. Thiol-disulfide redox buffers maintain a structure of immunoglobulin A that is essential for optimal in vitro binding to secretory component. Biochimica et Biophysica Acta 1998; 1429:265-274.) Dimeric and polymeric IgA containing both J chain and secretory component is again purified from the mixture by immobilized metal ion affinity chromatography, such as ion-exchange and size exclusion chromatography and/or ultrafiltration as described in Lullau et al., 1996; Corthesy, 1997; and Crottet et al., 1999; as performed by those of skill in the art of protein purification.

Purified dimeric and polymeric secretory IgA containing recombinant human secretory component is optionally stabilized for example by the addition of human serum albumin to a final concentration of 5%. The presence of the human secretory component in the compositions of the invention leads to doses of immunoglobulin A which are physiologically effective whereas compositions without secretory component are not. Additionally, this invention specifies an industrial method for the manufacture of polyclonal human secretory IgA comprised of recombinant human secretory component plus natural human plasma-derived IgA dimers and higher polymers which would not otherwise be obtainable in quantities sufficient for commercial medicinal use.

It has been previously found that it is possible to separate recombinant proteins from cell supernatants by producing such proteins with histidine affinity tags or other of the aforementioned affinity tags. The recombinant-protein-containing cell supernatants are passed through nickel bound solid support resins. The histidine or other tag adheres to the nickel or other suitable tag specific binding moiety and is retained while the unwanted proteins are washed therethrough. In the present invention the tagged secretory immunoglobulin protein is then recovered by eluting with an imidazole buffer in the case in which there is an amide-metal bond between the target protein and resin (Block H et al 2009).

The mixture of histidine tagged secretory IgA and residual plasma proteins is buffer exchanged into a binding buffer containing low concentrations of imidazole (≤40 mM). Another release agent operative to exchange histidine tagged secretory IgA or secretory IgM illustratively includes: (1) ethylene diamine tetraacetic acid (EDTA) at 10 mM and (2) an elution buffer of pH 5.5 or lower. Typical binding buffer imidazole concentrations range from 0.1 to 40 millimolar (mM). It is appreciated that the initial binding buffer pH is somewhat variable and readily discerned for a given chemical structure of buffer and concentration through routine experimentation. The chromatography medium operative herein is selected to be stable in the presence of the binding buffer and able to separate histidine tagged secretory IgA. Exemplary of these metal-bound solid support resins are nickel, cobalt and zinc immobilized on crosslinked, beaded-form of agarose (SEPHAROSE®). In a preferred embodiment, the affinity medium is washed in a wash buffer containing from 0 to 40 mM imidazole to remove unbound monomeric IgA and other non-specifically bound residual proteins. The bound histidine tagged secretory IgA is recovered using an elution buffer of a higher imidazole concentration (e.g., 100 to 1000 mM). With successive elutions, separation of monomeric from histidine tagged secretory component bound dimeric IgA is exacted. It is appreciated that the inventive process is amenable to scaling to produce quantities sufficient to treat numerous subjects. It is appreciated that similar selective binding pairs is achieved between other inventive tagged secretory component containing immunoglobulin proteins and resins are conventional to the art for each of the aforementioned tags.

By way of a specific example, the binding and wash buffers are 50 mM NaH₂PO₄, 300 mM NaCl, and 20 mM imidazole that is adjusted to pH 8. The mixture of IgA monomer and secretory IgA is dissolved in that buffer. The elution buffer is identical to the binding buffer with the exception that the imidazole is at a higher concentration, e.g., 100 to 1000 mM.

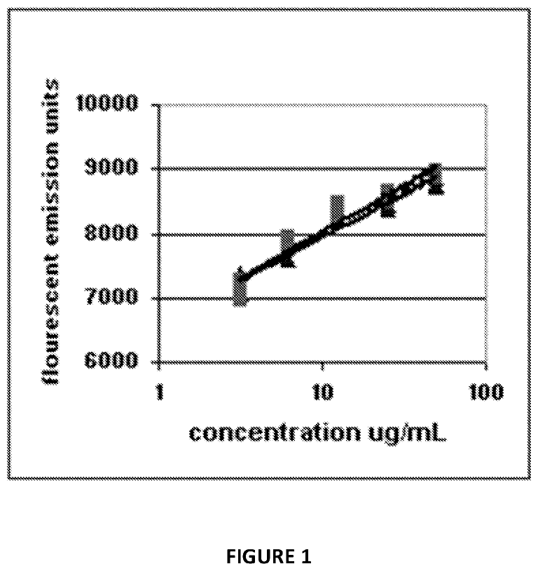

The remaining histidine tagged secretory IgA is then eluted from the divalent immobilized metal resin with the elution buffer according to conventional techniques and conditions that include an exemplary basic pH of for examples 8 to 10, see FIGS. 1.

Purified secretory IgA containing histidine tagged secretory component is stabilized in some embodiments for example by the addition of human serum albumin to a final concentration of 5% total weight albumen.

In another embodiment, the tag is removed from the recovered secretory IgA and native secretory IgA is available for usage as a medicament. For a histidine tagged secretory IgA a procedure for tag removal is known to the art (Kopera E et al 2012).

In summary, the inventive process is the addition of tagged amino terminus secretory component in either recombinant or post expression tagging to a mixture of plasma derived IgA monomers and dimers, in which the tagged secretory component combines with the IgA dimer forming secretory IgA and allows recovery of the newly formed secretory IgA by adhesion to immobilized divalent metal ions or other solid phase moiety, and subsequent elution therefrom.

In inventive embodiments, an affinity tag is located at the amino end (N-terminus) of the secretory component molecule. This tag position as shown in FIG. 2 allows sterically free access to the nickel affinity column after the secretory component has been joined to the IgA dimer forming secretory IgA and facilitates the recovery of the entire secretory IgA molecule using the affinity tag. In still other embodiments, the mature, N-terminus affinity tag, such as a histidine tag, has a capping amino acid, dipeptide, or 3 to 9 amino acid oligopeptide. The cap in some instance is a residue from cleavage of a signaling sequence.

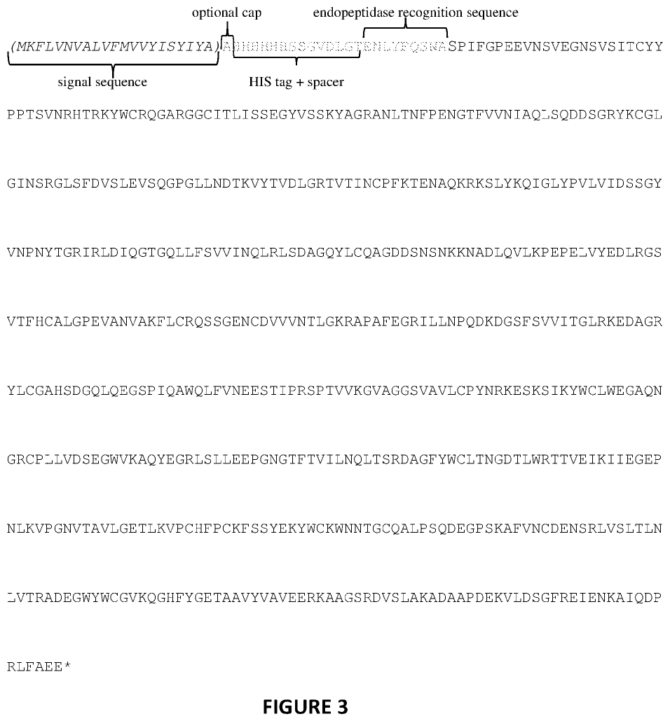

The amino acid sequence of the native secretory component is provided (SEQ ID. NO. 15) and shown with an additional N-terminus sequence in FIG. 3. It is appreciated that a FLAG sequence as detailed above with respect to a N-terminus tag is also operative herein. In some inventive embodiments, a spacer is provided intermediate between the polyhistidine sequence and the N-terminus of the native secretory component. It is appreciated that the spacer is highly variable and functions to insure exposure of the polyhistidine from the folded IgA-bound secretory component. A spacer, if present, has a length of between 2 and 20 residues. An exemplary polyhistidine spacer secretory component is provided (SEQ ID. NO. 16). In still other embodiments, an endopeptidase recognition sequence is provided as part of the spacer and adjacent to the native secretory component. The endopeptidase recognition sequence provides a cleavage site to optionally remove the affinity tag, and any optional spacer from the fusion protein to yield a native IgA-bound secretory component or alternatively provide a situs for binding for additional stages of purification. An exemplary endopeptidase recognition sequence operative herein is Tobacco Etch Virus (TEV) nuclear-inclusion-a endopeptidase recognition sequence. TEV protease is known to be a highly sequence-specific cysteine protease. Other endopeptidase recognition sequences operative herein illustratively include trypsin, factor Xa, elastase, chymotrypsin, enterokinase recognition sequence (A. Hillar), and collagenase.

In still other inventive embodiments, a recombinant IgA secretory component is provided with a signal sequence. The signal sequence functions to enhance expression from transfected cells. For nonlytic insect cell recombinant IgA secretory component expression signal sequences operative herein include honeybee melittin, or sequences derived from the major envelope proteins from nuclear polyhedrosis viruses such as AcNPV or OpNPV (Brown et al., 2011). For expression and secretion from mammalian cell culture signal sequences may be rat PAM (ZH Jiang et al.) or human cyclo-oxygenase 2 (Venkatesan, et al., 2021). An exemplary honeybee melittin-polyhistidine-spacer-IgA secretory component is provided (SEQ ID. NO. 18). In some inventive embodiments, the cleavage of the signal sequence is partial thereby leaving a cap adjacent to the affinity tag. An alanine cap is shown in FIG. 3 and SEQ ID. NO. 17.

Without intending to be bound to a particular theory, the signal sequence is cleaved off while the protein is still in the lumen of the endoplasmic reticulum of the expressing cell. The mature protein of FIG. 3 begins with an alanine N-terminus which is part of the signal sequence that remains after cleavage. This is followed directly by the affinity tag, e.g. the His tag. There is a spacer and then the Tobacco Etch Virus (TEV) nuclear-inclusion-a endopeptidase recognition sequence. TEV protease is noted to be a highly sequence-specific cysteine protease.

The invention further embraces variants and equivalents which are substantially homologous to secretory component and still retain the ability to selectively bind polymeric IgA, IgM, or both. These can contain, for example, conservative substitution mutations, i.e., the substitution of one or more amino acids by similar amino acids. For example, conservative substitution refers to the substitution of an amino acid with another within the same general class such as, for example, one acidic amino acid with another acidic amino acid, one basic amino acid with another basic amino acid, or one neutral amino acid by another neutral amino acid.

The secretory component of the present invention can be recombinant secretory component or synthetic secretory component retaining binding properties to IgA or IgM. It will be recognized in the art that some amino acid sequences of the invention can be varied without significant effect of the structure or function of the protein (patent application????????). the invention further includes variations of the secretory component which show substantial activity; such mutants include deletions, insertions, inversions, repeats, and type substitutions. Secretory component mutants operable herein illustratively include amino acid substitutions relative to SEQ ID NO: 16. Other sequence mutations operative herein are detailed in Stadtmueller et al. It is appreciated that other mutations at different amino acid sites are similarly operable. It is further appreciated that mutation of the conserved amino acid at any particular site is preferably mutated to glycine or alanine. It is further appreciated that mutation to any neutrally charged, charged, hydrophobic, hydrophilic, synthetic, non-natural, non-human, or other amino acid is similarly operable.

Modifications and changes are optionally made in the structure (primary, secondary, or tertiary) of the secretory component protein which are encompassed within the inventive compound that may or may not result in a molecule having similar characteristics to the exemplary polypeptides disclosed herein. It is appreciated that changes in conserved amino acid bases are most likely to impact the activity of the resultant protein. However, it is further appreciated that changes in amino acids operable for receptor interaction, resistance or promotion of protein degradation, intracellular or extracellular trafficking, secretion, protein-protein interaction, post-translational modification such as glycosylation, phosphorylation, sulfation, and the like, may result in increased or decreased activity of an inventive compound while retaining some ability to alter or maintain a physiological activity. Certain amino acid substitutions for other amino acids in a sequence are known to occur without appreciable loss of activity.

In making such changes, the hydropathic index of amino acids are considered. According to the present invention, certain amino acids can be substituted for other amino acids having a similar hydropathic index and still result in a polypeptide with similar biological activity. Each amino acid is assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

Without intending to be limited to a particular theory, it is believed that the relative hydropathic character of the amino acid determines the secondary structure of the resultant polypeptide, which in turn defines the interaction of the polypeptide with other molecules. It is known in the art that an amino acid can be substituted by another amino acid having a similar hydropathic index and still obtain a functionally equivalent polypeptide. In such changes, the substitution of amino acids whose hydropathic indices are within.+−0.2 is preferred, those within.+−0.1 are particularly preferred, and those within.+−0.05 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val). (Lys: Arg), (Met: Leu, Tyr), (Ser: Thr), (Thr: Ser), (Tip: Tyr), (Tyr: Trp, Phe), and (Val: Ile, Leu).

The secretory component and analogs can be further modified to contain additional chemical moieties not normally part of the protein. Those derivatized moieties can improve the solubility, the biological half-life, absorption of the protein, or binding affinity. The moieties can also reduce or eliminate any desirable side effects of the proteins and the like. An overview for those moieties can be found in Remington's Pharmaceutical Sciences, 20th ed., Mack Publishing Co., Easton, PA (2000).

Recombinant secretory component modified as detailed above with respect to IgA, it is appreciated to be applicable to purification of secretory IgM through either N- or C-terminus modification.

Plasma IgM can be recovered from the byproducts of the production of intravenous immunoglobulin. An example of such a byproduct is Cohn fraction III precipitate. The IgM is most easily solubilized from Cohn fraction III precipitate by 20 mM sodium acetate. Other plasma proteins are similarly solubilized along with the IgM. The plasma IgM in this protein mixture is covalently bound to recombinant histidine tagged secretory component in vitro forming secretory IgM within the protein mixture. The secretory IgM is now tagged by virtue of the divalent bonding of the tagged secretory component to the IgM. The novel method of obtaining purified secretory IgM that is thus formed is to remove the secretory IgM that is now tagged by affinity binding of the tag to an immobilized nickel⁺² or other divalent metal ion or other suitable binding moiety conventional to the art that is part of a resin column. The resultant semisynthetic secretory IgM has utility, for example, as a treatment of Clostridium difficile associated diseases such as Clostridium difficile colitis, pseudomembranous colitis, necrotizing enterocolitis, and antibiotic associated diarrhea and in particular to secretory immunoglobulin M compositions administered in the form of pharmaceutical compositions.

In another embodiment, the tag is removed from the recovered secretory IgM and native secretory IgM is available for usage as a medicament. For a histidine tagged secretory IgM a procedure for tag removal is known to the art (Kopera E et al 2012). In other embodiments, the spacer is also removed to result in native secretory IgM, regardless of whether a portion of the spacer is functional as an endopeptidase recognition sequence.

Thus, an inventive process provides the addition of peptide tagged secretory component to a mixture of plasma derived IgM and other plasma proteins, in which the peptide tagged secretory component combines with the IgM forming secretory IgM and allows recovery of the newly formed secretory IgM by adhesion to moieties on the solid phase support resin to which the peptide binds, and subsequent elution therefrom using an elution buffer.

In still other embodiments, an IgA is combined with pasteurized human milk or with human milk prepared in a bioreactor (Deng M, 2022).

The secretory IgA antibodies may be administered alone, or with various pharmaceutical adjuvants.

These compositions optionally contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example, sugars, sodium chloride, and the like. Prolonged residence in the intestinal lumen of the IgA can be brought about by the use of agents delaying absorption, for example, aluminum monostearate and gelatin.

Solid dosage forms for oral administration include capsules, tablets, pills, powders, and granules. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, e. carboxymethylcellulose, alginates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethylene glycols, and the like.

Solid dosage forms such as tablets, dragees, capsules, pills, and granules can be prepared with coatings and shells, such as enteric coatings and others well known in the art; as detailed, for example in U.S. Pat. No. 4,017,647; 4385078; 4518433; and 4556552.

Such solid dosages may contain opacifying agents and can also be of such composition that they release the active compound or compounds in a certain part of the intestinal tract in a delayed manner. Examples of embedding compositions which can be used are polymeric substances and waxes. The active compounds can also be in microencapsulated form, if appropriate, with one or more of the above-mentioned excipients.

Liquid dosage forms for oral administration include pharmaceutically acceptable emulsions, solutions, suspensions, syrups, and elixirs. A liquid dosage form consists of secretory IgA disolved in a 250 mM glycine solution in physiologic saline. In addition to the active compounds, the liquid dosage forms may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl alcohol, benzyl benzoate, propylene glycol, 1,3-butyleneglycol, dimethylformamide, oils, in particular, cottonseed oil, groundnut oil, corn germ oil, olive oil, castor oil and sesame oil, glycerol, tetrahydrofurfuryl alcohol, polyethylene glycols and fatty acid esters of sorbitan or mixtures of these substances, and the like.

Besides such inert diluents, the composition can also include adjuvants, such as wetting agents, emulsifying and suspending agents, sweetening, flavoring, and perfuming agents.

Suspensions, in addition to the active compounds, may contain suspending agents, as for example, ethoxylated isostearyl alcohols, polyoxyethylene sorbitol and sorbitan esters, microcrystalline cellulose, aluminum or other metal hydroxides, bentonite, agar-agar and tragacanth, or mixtures of these substances, and the like.

Since the effect of the IgA antibodies is dependent on their reaching the small intestine, preferred tablets or capsules are enteric coated. Alternatively, the active IgA antibodies can themselves be microencapsulated prior to formulation. Preparation of microcapsules of IgA antibody as well as preparation of enteric coated tablets or capsules can be achieved by conventional methods as detailed above.

It is appreciated that the therapeutic amount of sIgA depends on the form thereof, with forms subject to gastrointestinal degradation requiring larger doses. Typically amounts of sIgA from about 0.005 mg to 50 grams per day are used and preferably, 1 mg to 40 grams per day. Generally, secretory IgA are each independently effective as a treatment when provided to the patient at about 10 grams per day. Forms of IgA that are prone to gastrointestinal degradation are typically effective in doses increased by at least 80% relative to secretory forms. For example, about 5 grams of secretory IgA could be given to a subject per day in a single dose or in divided doses 3 to 4 times per day. Preferably, multiple doses are administered with meals likely containing food allergens. It is appreciated that a physician can readily adjust the doses of the IgA to be administered based on the subject response to treatment. Many factors are considered in dose adjustments. Dosages of secretory IgA envisioned by the present invention and considered to be therapeutically effective will range from between about 5 mg to 5 g. However, it is to be understood that doses can readily be adjusted to provide appropriate amounts of the IgA antibody.

The invention is distinguished from the prior art by the derivation of its dimeric and polymeric IgA component from pooled healthy human plasma. It is further distinguished by the conjugation of the dimeric and polymeric IgA components with amino terminus histadine affinity tagged recombinant human secretory component which is required for the normal activity of secretory IgA in the intestines. Importantly, this invention specifies an industrial method for the manufacture of polyclonal semisynthetic human secretory IgA which is not otherwise obtainable in amounts suitable for widespread medicinal use.

The invention is further described by reference to the following detailed examples, with exemplary process methodologies described below. These examples are not meant to limit the scope of the invention that has been set forth in the foregoing description. Variations within the concepts of the invention are apparent to those skilled in the art.

### EXAMPLES

### Example 1

Dimeric IgA is obtained by affinity purification from pooled healthy human plasma and conjugated with recombinant amino terminus histadine affinity tagged human secretory component, produced according to FIG. 3, to forming secretory IgA. The secretory IgA is stabilized by the addition of human serum albumin to a final concentration of 5%. The final solution is adjusted to a therapeutic dose of 100 mg secretory IgA daily. The secretory IgA is administered daily to an infant suffering with necrotizing enterocolitis. One week after initiation of treatment, the necrotizing enterocolitis sufferer experiences diminution of his/her physiological abnormalities.

### Example 2

The process of Example 1 is repeated with the secretory IgA administered enterically, at a higher daily dose of 1 g to achieve a similar result.

### REFERENCES

Aoyama K, Chiba J. Separation of different molecular forms of mouse IgA and IgM monoclonal antibodies by high-performance liquid chromatography on spherical hydroxyapatite beads. J Immunol Methods. 1993;162(2):201-10.

Bacon A. E. 3rd, Fekety R. Immunoglobulin G directed against toxins A and B of Clostridium difficile in the general population and patients with antibiotic-associated diarrhea. Diagn. Microbiol. Infect. Dis. 1994; 18:205-209.

Barroso L. A., Wang S. Z., Phelps C. J., Johnson J. L., Wilkins T. D. Nucleotide sequence of Clostridium difficile toxin B gene. Nucleic Acids Res. 1990; 18:4004.

Berzofsky J. A., Berkower I. J., Epstein S. L., Monoclonal Antibodies in Chapter 12, Antigen-Antibody Interactions and Monoclonal Antibodies, pp. 455-465 in Fundamental Immunology, Third Edition, W. E. Paul (ed), Raven Press, NY 1993. Berzofsky et al., Fundamental Immunology, Third Edition, 1993, pp 455-462.

Block H, Maertens B, Spriestersbach A, Brinker N, Kubicek J, Fabis R, Labahn J, Schafer F. Immobilized-Metal Affinity Chromatography (IMAC): A Review. CHAPTER 27 in Methods in Enzymology, Volume 463, 2009.

Boesman-Finkelstein M., Walton N. E., Finkelstein R. A. Bovine lactogenic immunity against cholera toxin-related enterotoxins and Vibrio cholerae outer membranes. Infect. Immun. 1989; 57:1227-1234.

Brown, W. C., DelProposto, J., Rubin, J. R., Lamiman, K., Carless, J. and Smith, J., New ligation-independent cloning vectors compatible with a high-throughput platform for parallel construct expression evaluation using baculovirus-infected insect cells, Protein Expr. Purif.,2011:77, 34-45.

Brussow H., Hilpert H., Walther I., Sidoti J., Mietens C., Bachmann P. Bovine milk immunoglobulins for passive immunity to infantile rotavirus gastroenteritis. J. Clin. Microbiol. 1987; 25:982-986.

Chiari E. F., Weiss W., Simon M. R., Kiessig S. T., Pulse M., Brown S. C., Gerding H. R., Mandago M., Gisch K., von Eichel-Streiber C., Oral Immunotherapy with Human Secretory IgA Improves Survival in the Hamster Model of Clostridioides difficile Infection, J Infect. Dis. 2021; October 28;224 (8): 1394-1397.

Cicalese L, Duerr R H, Nalesnik M A, Heeckt P F, Lee K K. Schraut W H. Decreased mucosal IgA levels in ileum of patients with chronic ulcerative colitis. Dig Dis Sci. 1995April;40 (4): 805-11.

Cohn E. J., Strong L. E., Hughes W. L., Jr., Mulford D. J., Ashworth J. N., Melin M., Taylor H. L., Preparation and Properties of Serum and Plasma Proteins IV. A System for the Separation into Fractions of the Protein and Lipoprotein Components of Biological Tissues and Fluids, J. Am. Chem. Soc. 1946; 68;459-475.

Collins S M, Denou E, Verdu E F, Bercik P. The putative role of the intestinal microbiota in the irritable bowel syndrome. Digestive and Liver Disease 41 (2009) 850-853

Cone L. A., Lopez C., Tarleton H. L., Jodoin D., Taylor M., Gade-Andavolu R., Dreisbach L. P. A durable response to relapsing Clostridium difficile colitis may require combined therapy with high-dose oral vancomycin and intravenous immune globulin. Infect. Dis. Clin. Pract. 2006; 14:217-220.

Corthesy B., Recombinant Secretory IgA for Immune Intervention Against Mucosal Pathogens, Biochem. Soc. Trans. 1997, 25;471-475.

Corthier et al., Emergence in Gnotobiotic Mice of Nontoxinogenic Clones of Clostridium difficile from a Toxinogenic One, Infection and Immunity, June 1988, pp. 1500-1504.

Corthier et al., Protection Against Experimental Pseudomembranous Colitis in Gnotobiotic Mice by Use of Monoclonal Antibodies Against Clostridium difficile Toxin A, Infection and Immunity, March 1991, pp. 1192-1195.

Crottet P., Cottet S., Corthesy B., Expression, Purification and Biochemical Characterization of Recombinant Murine Secretory Component, A Novel Tool in Mucosal Immunology, Biochem. J. 1999, 341;299-306.

Caubet J C, Nowak-Węgrzyn A. Current understanding of the immune mechanisms of food protein-induced enterocolitis syndrome. Expert Rev Clin Immunol. 2011 May;7(3):317-27.

Delacroix D. L., Hodgson H. J., McPherson A., Dive C., Vaerman J. P. Selective transport of polymeric immunoglobulin A in bile. Quantitative relationships of monomeric and polymeric immunoglobulin A, immunoglobulin M, and other proteins in serum, bile, and saliva. J. Clin. Invest. 1982 August;70(2):230-41

Delacroix D. L., Elkom K. B., Geubel A. P., Hodgson H. F., Dive C., Vaerman J. P. Changes in size, subclass, and metabolic properties of serum immunoglobulin A in liver diseases and in other diseases with high serum immunoglobulin A. J. Clin. Invest. 1983 February;71(2):358-67.

Deng M, Lv X, Liu L, Li J, Du G, Chen J, and Liu Y. Efficient Bioproduction of Human Milk Alpha-Lactalbumin in Komagataella phaffii J. Agric. Food Chem. 2022, 70, 8, 2664-2672.

Donaldson G P, Ladinsky M S, Yu K B, Sanders J G, Yoo B B, Chou W C, Conner M E, Earl A M, Knight R, Bjorkman P J, Mazmanian SK Gut microbiota utilize immunoglobulin A for mucosal colonization. Science. 2018 May 18;360(6390):795-800.

Dove C. H., Wang S. Z., Price S. B., Phelps C. J., Lyerly D. M., Wilkins T. D. and Johnson J. L.; Lyerly et al. Molecular characterization of the Clostridium difficile toxin A gene. Infect. Immun. 1990; 58:480-488.

Ehrich et al., Production of Clostridium difficile Antitoxin, Infection and Immunity, June 1980, pp. 1041-1043.

Eibl M M, Wolf H M, Fürnkranz H, Rosenkranz A. Prophylaxis of necrotizing enterocolitis by oral IgA-lgG: review of a clinical study in low birth weight infants and discussion of the pathogenic role of infection. J Clin Immunol. 1990 November; 10(6 Suppl):72S-77S; discussion 77S-79S. doi: 10.1007/BF00918694. PMID: 2081793; PMCID: PMC7086514.

Eibl M M. Wolf H M, Fürnkranz H, Rosenkranz A. Prevention of necrotizing enterocolitis in low-birth-weight infants by IgA-IgG feeding. N Engl J Med. 1988 Jul. 7;319(1):1-7. doi: 10.1056/NEJM198807073190101. PMID: 3288866.

Fayer R., Guidry A., Blagburn B. L. Immunotherapeutic efficacy of bovine colostral immunoglobulins from a hyperimmunized cow against cryptosporidiosis in neonatal mice. Infect. Immun., 1990; 58:2962-2965.

Fadlallah J, El Kafsi H, Sterlin D, Juste C, Parizot C, Dorgham K, Autaa G, Gouas D, Almeida M, Lepage P, Pons N, Le Chatelier E, Levenez F, Kennedy S. Galleron N, de Barros J P, Malphettes M, Galicier L, Boutboul D, Mathian A, Miyara M, Oksenhendler E, Amoura Z, Doré J, Fieschi C, Ehrlich S D, Larsen M, Gorochov G. Microbial ecology perturbation in human IgA deficiency. Sci Transl Med. 2018 May 2;10 (439).

Frossard C P, Hauser C, Eigenmann P A. Antigen-specific secretory IgA antibodies in the gut are decreased in a mouse model of food allergy. J Allergy Clin Immunol. 2004 August;114(2):377-82.

Gerding et al., clostridium difficile-Associated Diarrhea, Archives of Internal Medicine, vol. 146, January 1986, pp. 95-100.

Granato D A, Piguet P F. A mouse monoclonal IgE antibody anti bovine milk beta-lactoglobulin allows studies of allergy in the gastrointestinal tract. Clin Exp Immunol. 1986 March;63(3):703-10.

Gopalakrishna K P, Macadangdang B R, Rogers M B, Tometich J T, Firek B A, Baker R, Ji J, Burr AHP, Ma C, Good M. Morowitz M J, Hand T W. Maternal IgA protects against the development of necrotizing enterocolitis in preterm infants. Nat Med. 2019 July;25(7):1110-1115.

Harrison M, Kilby A, Walker-Smith JA, France NE, Wood CB. Cows' milk protein intolerance: a possible association with gastroenteritis, lactose intolerance, and IgA deficiency. Br Med J. 1976 Jun. 19;1 (6024):1501-4.

Harumi J. Non-IgE Mediated Food Allergy. Inflammation & Allergy-Drug Targets, 2008, 7, 3, 173-180 (8).

Hermanson G T. Bioconjugate Techniques.Academic Press, San Diego 1996.

Hillar A, Otulakowski G, O Brodovich H. Purification and characterization of a recombinant rat prohaptoglobin expressed in baculovirus-infected Sf9 insect cells. Protein Expr Purif. 2007 October: 55 (2): 246-56. Epub 2007 Jun. 30. PMID: 17681809.

Hilpert H., Brussow H., Mietens C., Sidoti J., Lerner L., Werchau H. Use of bovine milk concentrate containing antibody to rotavirus to treat rotavirus gastroenteritis in infants. J. Infect. Dis. 1987; 156:158-166.

Jiang Z H, Yang Y H, Wu X F, Li B L, Wang D B. Efficient Secretion of Proteins Expressed from Insect Cells Directed by PAM Signal Peptide. Sheng Wu Hua Xue Yu Sheng Wu Wu Li Xue Bao (Shanghai). 1999;31 (1): 74-78. PMID: 12142920. Johnson S. et al. Infect. Immun. 1995; 63:3166-3173.

Jones R. M. L., Schweikart F., Frutiger S., Jaton J-C., Hughes G. J. Thiol-disulfide redox buffers maintain a structure of immunoglobulin A that is essential for optimal in vitro binding to secretory component. Biochimica et Biophysica Acta 1998; 1429:265-274.

Kabir S. Jacalin: a jackfruit (Artocarpus heterophyllus) seed-derived lectin of versatile applications in immunobiological research. J Immunol Methods 1998, 212:193-211

Kelly C P, Chetham S, Keates S, Bostwick E F, Roush A M, Castagliuolo I, LaMont J T, Pothoulakis C. Survival of Anti-Clostridium difficile Bovine Immunoglobulin Concentrate in the Human Gastrointestinal Tract. Antimicrob Agents Chemother. 1997 February;41(2):236-41.

Kelly et al., Clostridium difficile Colitis, New England Journal of Medicine, vol. 330, January 1994, pp. 257-262.

Kelly et al., Human Colonic Aspirates Containing Immunoglobulin A Antibody to Clostridium difficile Toxin A Inhibit Toxin A-Receptor Binding, Gastroenterology, vol. 102, No. 1, pp. 35-40.

Kohler G., Milstein C., Continuous Cultures of Fused Cells Secreting Antibody of Predetermined Specificity, Nature 1975; 256;495-497.

Kopera E, Belczyk-Ciesielska A, Bal W. Application of Ni(II)-assisted peptide bond hydrolysis to non-enzymatic affinity tag removal. PLOS One. 2012;7 (5): e36350. doi: 10.1371/journal.pone.0036350. Epub 2012 May 4.

Leung D. Y., Kelly C. P., Boguniewicz M., Pothoulakis C., LaMont J. T., Flores A. Treatment with intravenously administered gamma globulin of chronic relapsing colitis induced by Clostridium difficile toxin. J. Pediatr. 1991 April; 118(4(Pt 1)):633-637.

Levy M, Kolodziejczyk A A, Thaiss C A, Elinav E. Dysbiosis and the immune system. Nat Rev Immunol. 2017 April; 17(4):219-232.

Libby J. M., Jortner B. S., Wilkins T. D. Effects of the two toxins of Clostridium difficile in antibiotic-associated cecitis in hamsters. Infect. Immun. 1982 May; 36(2):822-829.

Lima et al., Effects of Clostridium difficile Toxins A and B in Rabbit Small and Large Intestine In Vivo and on Cultured Cells In Vitro, Infection and Immunity, March 1988, pp. 582-588.

Longet S, Miled S, Lötscher M, Miescher S M, Zuercher A W, Corthésy B. Human plasma-derived polymeric IgA and IgM antibodies associate with secretory component to yield biologically active secretory-like antibodies. J Biol Chem. 2013 Feb. 8;288:4085-94.

Louie T. J., Peppe J., Watt C. K., Johnson D., Mohammed R., Dow G., Weiss K., Simon S., John J. F. Jr., Garber G., Chasan-Taber S., Davidson D. M.; Tolevamer Study Investigator Group. Tolevamer, a novel nonantibiotic polymer, compared with vancomycin in the treatment of mild to moderately severe Clostridium difficile-associated diarrhea. Clin. Infect. Dis. 2006; 43:411-20.

Luellau, E., von Stockar, U., Vogt, S., Freitag, R. Development of a downstream process for the isolation and separation of monoclonal immunoglobulin A monomers, dimers and

polymers from cell culture supernatant, Journal of Chromatography A, 1998, 796:165-175.

Lullau E., Heyse S., Vogel H., Marison I., von Stockar U., Kraehanbuhl J-P., Corthesy B., Antigen Binding Properties of Purified Immunoglulin A Antibodies, J. Biol. Chem. 1996; 271:16300-16309.

Lyerly D. M., Krivan H. C., Wilkins T. D. Clostridium difficile: its disease and toxins. Clin. Microbiol. Rev. 1988; 1:1-18.

Lyerly D. M., Phelps C. J., Toth J., Wilkins T. D. Characterization of toxins A and B of Clostridium difficile with monoclonal antibodies. Infect. Immun. 1986; 54:70-76.

Lyerly D. M., Bostwick E. F., Binion S. B., Wilkins T. D. Passive immunization of hamsters against disease caused by Clostridium difficile by use of bovine immunoglobulin G concentrate. Infect. Immun. 1991; 59:2215-2218.

Lyerly D. M., Lockwood D. E., Richardson S. H., Wilkins T. D. Biological activities of toxins A and B of Clostridium difficile. Infect. Immun. 1982; 35:1147-1150.

Lyerly D. M., Saum K. E., MacDonald D. K., Wilkins T. D. Effects of Clostridium difficile toxins given intragastrically to animals. Infect. Immun. 1985; 47:349-352.

Mahe et al., Effect of Various Diets on Toxin Production by Two Strains of Clostridium difficile in Gnotobiotic Mice, Infection and Immunity, August 1987, pp. 1801-1805.

Martinez et al., Purification and Characterization of Clostridium sordellii Hemorrhagic Toxin and Cross-Reactivity with Clostridium difficile Toxin A (Enterotoxin), Infection and Immunity, May 1988, pp. Dec. 15, 1221.

McFarland et al., Nosocomial Acquisition of Clostridium difficile Infection, The New England Journal of Medicine, January 1989, pp. 204-210.

McFarland et al., Review of Clostridium difficile Associated Diseases, American Journal of Infection Control, vol. 14, No. 3, June 1986, pp. 99-104.

McPherson S., Rees C. J., Ellis R., Soo S. and Panter S. J. Intravenous Immunoglobulin for the Treatment of Severe, Refractory, and Recurrent Clostridium difficile Diarrhea. Diseases of the Colon & Rectum. 2006; 49 (5): 640-645.

Med. Letter Drugs Ther. 2006; 48:89-90,92.

Mietens C., Keinhorst H., Hilpert H., Gerber H., Amster H., Pahud J. J. Treatment of infantile E. coli gastroenteritis with specific bovine anti-E. coli milk immunoglobulins. Eur. J. Pediatr. 1979; 132:239-252.

Mitchell et al., Effect of Toxin A and B of Clostridium difficile on Rabbit Ileum and Colon, Gut, 1986, vol. 27, pp. 78-85.

Morris et al., Role of Surgery in Antibiotic-Induced Pseudomembranous Enterocolitis, The American Journal of Surgery, vol. 160, November 1990, pp. 535-539.

Nakajima A1, Vogelzang A, Maruya M, Miyajima M, Murata M1, Son A, Kuwahara T, Tsuruyama T, Yamada S, Matsuura M5, Nakase H, Peterson D A, Fagarasan S, Suzuki K IgA regulates the composition and metabolic function of gut microbiota by promoting symbiosis between bacteria. J Exp Med. 2018 Aug. 6;215(8):2019-2034.

Oncley J. L., Melin M., Richert D. A., Cameron J. W., Gross P. M., Jr., The Separation of the Antibodies, Isoagglutinins, Prothrombin, Plasminogen and B1-Lipoprotein into Subfractions of Human Plasma. J. Am. Chem. Soc. 1949; 71:541-550.

Possin M E, Morgan S, DaSilva D F, Tisler C, Pappas T E, Roberg K A, Anderson E, Evans M D, Gangnon R, Lemanske R F, Gern J E. The relationships among immunoglobulin levels, allergic sensitization, and viral respiratory illnesses in early childhood. Pediatr Allergy Immunol 2010:21: 990-996.

Pothoulakis C., LaMont J. T., Eglow R., Gao N., Rubins J. B., Theoharides T. C., Dickey B. F. Characterization of rabbit ileal receptors for Clostridium difficile toxin A. Evidence for a receptor-coupled G protein. J. Clin. Invest. 1991; 88:119-25.

Rogier, et al. Secretory antibodies in breast milk promote long-term intestinal homeostasis by regulating the gut microbiota and host expression (PNAS 2014:111 (8) 3074-3079.

Rothman et al., Differential Cytotoxic Effects of Toxins A and B Isolated from clostridium difficile, Infection and Immunity, November 1984, pp. 324-331.

Salcedo J. et. al. Gut 1997; 41:366-370.

Schwarze J, Cieslewicz G, Joetham A, L Sun L K, Sun W N, Chang T W, Hamelmann E, W. Gelfand E W. Antigen-specific Immunoglobulin-A Prevents Increased Airway Responsiveness and Lung Eosinophilia after Airway Challenge in Sensitized Mice. Am J Resp Crit Care Med 1998;158:519-525.

Shimoda M, Inoue Y, Azuma N, Kanno C. Local antibody response in Peyer's patches to the orally administered dietary protein antigen. Biosci Biotechnol Biochem. 1999 December;63(12):2123-9.

Simon M R, et al., Polyclonal antibody therapies for Clostridium difficile infection. Antibodies 2014;3:272-288.

Stadtmueller B. M., Huey-Tubman K. M, López C. J., Yang, Z., Hubbell W. L., Bjorkman P. J. The structure and dynamics of secretory component and its interactions with polymeric immunoglobulins. eLife 2006; 5: e10640.

Strong L. E., Blood Fractionation, pp. 576-602 in vol. 3, Kirk-Othmer Encyclopedia of Chemical Technology. Second Edition, H. F. Mark, J. J. McKetta, D. F. Othmer (eds), Interscience Publishers, NY 1963, pp. 576-602.

Stubbe H. et al. J. Immunol. 2000; 164:1952-1960.

Symersky J., Novak J., McPherson D. T., DeLucas L., Mestecky J. Expression of the recombinant human immunoglobulin J chain in Escherichia coli. Mol. Immunol. 2000; 37:133-140.

Tacket C. O., Losonsky G., Link H., Hoang Y., Guesry P., Hilpert H., Levine M. M. Protection by milk immunoglobulin concentrate against oral challenge with enterotoxigenic Escherichia coli. N. Engl. J. Med. 1988; 318:1240-3.

Tjellstrom B., Stenhammar L., Eriksson S., Magnusson K. E. Oral immunoglobulin A supplement in treatment of Clostridium difficile enteritis. Lancet 1993; 341(8846):701-702.

Trajkovski V, Ajdinski L, Spiroski M. Plasma concentration of immunoglobulin classes and subclasses in children with autism in the Republic of Macedonia: retrospective study. Croat Med J. 2004 December;45(6):746-9.

Trajkovski V, Ajdinski L. Spiroski M. Plasma concentration of immunoglobulin classes and subclasses in children with autism in the Republic of Macedonia: retrospective study. Croat Med J. 2004 December;45(6):746-9.

Triadafilopoulos et al., Differential Effects of Clostridium difficile Toxins A and B on Rabbit Ileum, Gastroenterology, 1987, vol. 93, pp. 273-279.

Tucker et al., Toxin A of Clostridium difficile Is a Potent Cytotoxin, Journal of Clinical Microbiology. May 1990, pp. 869-871.

Venkatesan A, Geng J, Kandarpa M, Wijeyesakere S J, Bhide A, Talpaz M. Pogozheva I D, Raghavan M. J, Cell Biol. 2021 Jul. 5;220(7):e202009179. doi: 10.1083/jcb.202009179.PMID: 33909030.

Vighi G, Marcucci F, Sensi L, G. Di Cara G, Frati F. Allergy and the gastrointestinal system. Clinical and Experimental Immunology, 153 (Suppl. 1): 3-6, 2008.

Vojdani A. Detection of IgE, IgG, IgA and IgM antibodies against raw and processed food antigens. Nutr Metab (Lond). 2009 May 12;6:22.

Walker A M, Kemp A S, Hill D J, Shelton M J. Features of transient hypogammaglobulinaemia in infants screened for immunological abnormalities. Arch Dis Child. 1994 March; 70 (3): 183-186.

Wang M, Takeda K, Shiraishi Y, Okamoto M, Dakhama A, Joetham A, Gelfand EW. Peanut-induced intestinal allergy is mediated through a mast cell-IgE-FcepsilonRI-IL-13 pathway. J Allergy Clin Immunol. 2010 August; 126(2):306-16, 316.e1-12.

Weltzin R., Traina-Dorge V., Soike K., Zhang J. Y., Mack P., Soman G., Drabik G., Monath T. P., Intranasal Monoclonal IgA Antibody against Respiratory Syncytial Virus Protects Rhesus Monkeys against Upper and Lower Respiratory Tract Infection. J. Infect. Dis. 1996; 174:256-261.

Weltzin R., Hsu S. A., Mittler E. S., Georgakopoulas K., Monath T. P., Intranasal Monoclonal Immunoglobulin A against Respiratory Synctial Virus Protects against Upper and Lower Respiratory Tract Infections in Mice. Antimicrob. Agents Chemother. 1994; 38:2785-2791.

Wilcox M. H. J. Antimicrob. Chemoth. 2004; 53:882-884.

Wolf H M, Eibl M M. The relevance of immunoglobulin in the prevention of necrotizing enterocolitis. Adv Exp Med Biol. 1991;310:381-9. doi: 10.1007/978-1-4615-3838-7_49.PMID: 1809014.

Wolf H M, Eibl M M. The anti-inflammatory effect of an oral immunoglobulin (IgA-IgG) preparation and its possible relevance for the prevention of necrotizing enterocolitis. Acta Paediatr Suppl. 1994;396:37-40. doi: 10.1111/j.1651-2227.1994.tb13240.x. PMID: 8086680.

Yang P-C, Wang C-S, An Z-Y. A murine model of ulcerative colitis: induced with sinusitis-derived superantigen and food allergen. BMC Gastroenterology 2005, 5:6.

Yoon MY, Yoon SS. Disruption of the Gut Ecosystem by Antibiotics. Yonsei Medical Journal. 2018, 59(1):4.

Yoshiyama Y., Brown W. R. Specific antibodies to cholera toxin in rabbit milk are protective against Vibrio cholerae-induced intestinal secretion. Immunology. 1987; 61:543-547.

Patent applications and publications mentioned in the specification are indicative of the levels of those skilled in the art to which the invention pertains. These applications and publications are incorporated herein by reference to the same extent as if each individual application or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

In the claims:

1. A recombinant secretory component comprising:
a secretory component having
an affinity tag bound to the amino-terminus.

2. The recombinant secretory component of claim 1 wherein said affinity tag is a histidine sequence of between 2 and 20 residues.

3. The recombinant secretory component of claim 1 further comprising a spacer peptide sequence intermediate between the amino-terminus and said affinity tag.

4. The recombinant secretory component of claim 1 wherein said spacer comprises an endopeptidase recognition sequence.

5. The recombinant secretory component of claim 1 further comprising a complete or residue of a signal sequence bound to terminal to said affinity tag.

6. The recombinant secretory component of claim 1 having one of SEQ ID Nos. 14, 15, or 16.

7. A process for inhibiting symptoms of, or preventing, necrotizing enterocolitis in a subject suffering therefrom, the process comprising:
administering orally to the subject suffering from necrotizing enterocolitis a purified polymeric secretory IgA comprising a recombinant human secretory component and human plasma derived IgA dimer and higher polymers; and
allowing sufficient time for said secretory IgA to inhibit symptoms of the necrotizing enterocolitis in the subject.

8. The process of claim 7 further comprising microencapsulating said prior to said administration.

9. The process of claim 7 wherein said secretory IgA is stabilized by the addition of human serum albumin prior to, or with said administration.

10. The process of claim 7 wherein said secretory IgA is stabilized by delivery with an antiacid.

11. The process of claim 7 wherein said secretory IgA is dissolved in an aqueous glycine solution.

12. The process of claim 7 wherein the secretory IgA is manufactured by an industrial process.

13. The process of claim 7 wherein the subject is a human infant.
